(19) United States
(12) Patent Application Publication
(10) Pub. No.: US 2010/0093739 A1
(43) Pub. Date: Apr. 15, 2010

(54) METHOD FOR DECREASING CAPILLARY PERMEABILITY IN THE RETINA

(76) Inventors: Romulus Kimbro Brazzell, Alpharetta, GA (US); Kenneth E. Green, Alpharetta, GA (US); Frances Elizabeth Kane, Cumming, GA (US); Peter Anthony Campochiaro, Baltimore, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/634,837

(22) Filed: Dec. 10, 2009

Related U.S. Application Data
(63) Continuation of application No. 10/512,505, filed on Jun. 3, 2005, now abandoned.
(60) Provisional application No. 60/376,829, filed on Apr. 30, 2002.

(51) Int. Cl. A61K 31/502; A61P 27/02

(52) U.S. Cl. 514/248

(57) ABSTRACT

Disclosed herein are methods for decreasing or attenuating an increase in capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject.

The blood-retinal barrier (BRB) consists of an inner component, comprised of the retinal capillary endothelial cells, and an outer component, comprised of retinal pigment epithelial cells. The BRB separates the retina from the efflux of the fenestrated vessels, primarily capillaries, of the choroid. The BRB is established by complexly arranged tight junctions between the barrier-forming cells and a paucity of endocytic vesicles within these cells. Both components of the BRB function by excluding blood-borne proteins and small, water-soluble non-electrolytes from the retina and by maintaining cell polarity through ionic and metabolic gradients. Increased permeability of the BRB and subsequent leakage can lead to macular edema, such as diabetic macular edema, the primary cause of loss of vision in early diabetic retinopathy.

In one aspect, the present invention relates to a method for decreasing capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject.

In a further aspect, the invention relates to a method for decreasing capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject, where the subject is suffering from clinically significant diabetic macular edema.

In a further aspect, the invention relates to a method for decreasing capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject, where the subject is suffering from diabetic macular edema which is not yet clinically significant.

In a further aspect, the invention relates to a method for decreasing capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject, where the subject is suffering from visual acuity loss due to diabetic macular edema.

In a further aspect, the invention relates to a method for decreasing capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject, where the subject is suffering from macular edema but does not also suffer from diabetes. Such macular edemas include those that are caused by, inter alia, branch retinal vein occlusion, pseudophakic or aphakic cystoid macular edema, intermediate uveitis, idiopathic retinal telangiectasia, vitreomacular traction syndrome, and dominantly inherited cystoid macular edema.

In another aspect, the invention relates to the practice of the abovementioned methods where the total effective amount is delivered to the subject in a single administration at a single time point during the day.

In another aspect, the invention relates to the practice of the abovementioned methods where the total effective amount is delivered to the subject in multiple administrations at multiple time points during the day.

In another aspect, the invention relates to the practice of the abovementioned methods where the effective amount is administered topically to the eye.

In another aspect, the invention relates to the practice of the abovementioned methods where the effective amount is administered intravitreally, subconjunctivally, periorbitally, or peribuibarly to the eye.

In another aspect, the invention relates to a method for treating a subject suffering from diabetic nephropathy or diabetic neuropathy comprising administering an effective amount of a composition comprising an amount of a phthalazine derivative or a salt thereof to a subject suffering from diabetic nephropathy or diabetic neuropathy to ameliorate at least one symptom of said diabetic nephropathy or diabetic neuropathy.

In still further aspects the invention relates to the use of a phthalazine derivative or a salt thereof in the manufacture of a medicament for decreasing or attenuating an increase in capillary permeability in the retina in a subject suffering from excessive or pathological capillary permeability in the retina;

the use of a phthalazine derivative or a salt thereof in the manufacture of a medicament for the treatment of clinically significant macular edema;

the use of a phthalazine derivative or a salt thereof in the manufacture of a medicament for the treatment of macular edema which is not clinically significant;

the use of a phthalazine derivative or a salt thereof in the manufacture of a medicament for the treatment of visual acuity loss, particularly if said visual acuity loss is associated or caused by an increase in capillary permeability in the retina or a clinically significant macular;

the use of a phthalazine derivative or a salt thereof in the manufacture of a medicament for the treatment of macular edema, not associated with diabetes;

the use a phthalazine derivative or a salt thereof in the manufacture of a medicament for the treatment of branch retinal vein occlusion, pseudophakic cystoid macular edema, aphakic cystoid macular edema, intermediate uveitis, idiopathic retinal telangiectasia, vitreomacular traction syndrome, or dominantly inherited cystoid macular edema; and

the use of a phthalazine derivative or a salt thereof in the manufacture of a medicament for the treatment of diabetic neuropathy.

As used herein, the term “clinically significant macular edema” means that a diabetic subject is suffering from one or more of (1) retinal edema within 500 micrometers from the center of the fovea (2) hard exudates within 500 micrometers of the fovea, if associated with adjacent retinal thickening, which thickening may be outside the 500 micrometer limit or (3) retinal edema that is within one disc area, i.e., 1500 micrometers, any part of which is within one disc diameter of the center of the fovea.

As used herein, a subject in need of treatment who is suffering from macular edema which is not clinically significant is a subject who is suffering from an increase in capillary permeability in the retina where the increase in capillary permeability is detectable using methods familiar to those of ordinary skill in the art, including ophthalmoscopy stereoscopic fundus photography and fluorescein angiography.

As used herein, the term “visual acuity loss” means reduction in the clarity of central vision due to increased retinal capillary permeability, such as that found in subjects diagnosed with diabetic macular edema.

There is provided in accordance with the present invention a method for decreasing capillary permeability in the retina of a subject in need of such treatment. The method comprises the step of administering an amount of a phthalazine derivative or a salt thereof to a subject suffering from excessive or pathological capillary permeability in the retina, more specifically capillary permeability in the retina structures that comprise the BRB, effective to decrease the capillary permeability.

The methods of the present invention use a composition containing a phthalazine derivative. Surprisingly, it has namely been found that certain phthalazine derivatives, in particular the compounds disclosed in U.S. Pat. No. 6,258,812 are useful for decreasing capillary permeability in the retina.

The pharmaceutical composition of the present invention which contains a compound disclosed in U.S. Pat. No. 6,258,812 as the active ingredient can be administered enterally, nasally, buccally, rectally, topically, orally, and parenterally, e.g., intravenous, intramuscular, periorbital, peribulbar, intravitreal, subconjunctival or subcutaneous administration, to decrease capillary permeability in mammalian subjects, especially humans. The compositions may contain the active ingredient alone or, preferably, the active ingredient along with a pharmaceutically acceptable carrier. The effective dosage of the active ingredient depends on the type of targeted disease, as well as the species, age, weight and physical condition of the subject, pharmacokinetic data, and the mode of administration.

Preferred total daily doses in humans are, e.g., between about 1 and about 4000 milligrams, between about 100 and about 2000 milligrams, between about 300 and about 2000 milligrams, between about 500 and about 2000 milligrams, and between about 1000 and about 2000 milligrams, e.g., about 1250 or about 1500 or about 1750 mg. Dosages can be repeated on a daily basis as necessary to achieve the desired decrease in capillary permeability. Daily doses can be administered at one time point, or can be divided with the total daily dose being administered over several time points during the day. It is preferred to administer the total daily dose in two individual doses, when administering the drug orally.

When administered topically or intravitreally, an effective dosage falls within the ranges set out for oral administration.

The compounds useful in the methods of the invention are administered in an amount effective to decrease capillary permeability in the retina of a subject suffering from excessive or pathological capillary permeability in the retina. Suitable pharmaceutical compositions may have from about 1% to about 95% of the active ingredient. Suitable unit dose forms include coated and uncoated tablets, ampoules, vials, suppositories, or capsules. Other suitable dosage forms include injectables, intraocular devices, intravitreal devices, ointments, creams, pastes, foams, tinctures, eye-drops, eye ointments, gels, oral drops, sprays, dispersions and the like. The pharmaceutical compositions useful in the methods of the present invention are prepared in a manner known in the art, for example, by means of conventional mixing, granulating, coating, dissolving or lyophilizing processes.

Aqueous solutions of the active ingredient, and also suspensions or dispersions, especially isotonic aqueous solutions, dispersions or suspensions are also useful in the practice of the invention. Suitable useful pharmaceutical compositions containing the active ingredient may have carriers, e.g., mannitol and starch, preservatives, stabilizers, wetting agents, emulsifiers, solubilizers, salts for regulating osmotic pressure, buffers and the like. The compositions are prepared in a manner known in the art, for example by means of conventional dissolving and lyophilizing processes. A solution or suspension form of the composition may contain viscosity-increasing agents, e.g., sodium carboxymethylcellulose, carboxymethylcellulose, dextran, polyvinylpyrrolidone, and gelatins; and solubilizers, e.g., Tween 80 (polyoxyethylene(20)sorbitan mono-oleate; trademark of ICI Americas, Inc, USA).

Suitable carriers include fillers, e.g., sugars, for example lactose, saccharose, mannitol or sorbitol; cellulose preparations; calcium phosphates, e.g., tricalcium phosphate and calcium hydrogen phosphate; binders, e.g., starches, methylcellulose, hydroxypropyl methylcellulose, sodium carboxymethylcellulose and polyvinylpyrrolidone; and, if desired, disintegrators, e.g., starches, crosslinked polyvinylpyrrolidone, alginic acid or salts thereof. Additional suitable excipients are flow conditioners and lubricants, e.g., silicic acid, talc, stearic acid and salts thereof, such as magnesium or calcium stearate, polyethylene glycol, and derivatives thereof.

In addition to the active ingredients, pharmaceutical compositions useful in the practice of the presently claimed methods may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Suitable phthalazine derivatives are kinase inhibitor active ingredients and are preferably selected from the compounds described in U.S. Pat. No. 6,258,812.

Particularly preferred are the compounds of formula I:

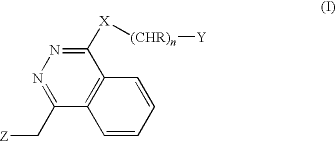

wherein
n is 0 to 2,
R is H or lower alkyl, in particular C₁-C₄-alkyl, e.g. n-butyl, sec-butyl, tert-butyl, n-propyl, isopropyl, or especially methyl or also ethyl;
X is imino, oxa, or thia;
Y is aryl, e.g. an aromatic moiety having 6 to 14 carbon atoms, especially phenyl, naphthyl, fluorenyl or phenanthrenyl, said moieties being unsubstituted or substituted by one or more, preferably up to three, especially one or two substituents, preferably selected from amino, mono- or disubstituted amino,'halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkylsulfonyl, and alkylphenylsulfonyl, lower alkenyl, such as ethenyl, phenyl, lower alkylthio, such as methylthio, lower alkanoyl, such as acetyl, lower alkylmercapto, such as methylmercapto (—S—CH₃), halogen-lower alkylmercapto, such as trifluoromethylmercapto (—S—CF₃), lower alkylsulfonyl, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy; and
Z is unsubstituted pyridyl, preferably 3- or even more preferably 4-pyridy; or substituted pyridyl, preferably 3- or 4-pyridyl which is substituted by one or two substituents, in particular selected from lower alkyl, preferably methyl, ethyl; halogen preferably chloro, fluoro, bromo; lower alkyl halides preferably trifluoromethyl; lower alkoxy preferably methoxy, ethoxy; hydroxy; cyano; amino, N-lower alkylamino, N,N-di-lower alkylamino, especially 4pyridyl substituted by methyl, chloro, fluoro, trifluoromethyl or methoxy;
   or an N-oxide of the defined compound of formula I, wherein one or more N atoms carry an oxygen atom.

Preferred examples of such compounds include:

1-(4-Chloroanilino)-4-(4-pyridylmethyl)phthalazine;

1-(3-Chloroanilino)-4-(4-pyridylmethyl)phthalazine;

1-Anilino-4-(4-pyridylmethyl)phthalazine;

1-Benzylamino-4-(4-pyridylmethyl)phthalazine;

1-(4-Methoxyanilino)-4-(4-pyridylmethyl)phthalazine;

1-(3-Benzyloxyanilino)-4-(4-pyridylmethyl)phthalazine;

1-(3-Methoxyanilino)-4-(4-pyridylmethyl)phthalazine;

1-(2-Methoxyanilino)-4-(4-pyridylmethyl)phthalazine;

1-(4-Trifluoromethylanilino)-4-(4-pyridylmethyl)phthalazine;

1-(4-Fluoroanilino)-4-(4-pyridylmethyl)phthalazine;

1-(3-Hydroxyanilino)-4-(4-pyridylmethyl)phthalazine;

1-(4-Hydroxyanilino)-4-(4-pyridylmethyl)phthalazine;

1-(3-Aminoanilino)-4-(4-pyridylmethyl)phthalazine;

1-(3,4-Dichloroanilino)-4-(4-pyridylmethyl)phthalazine;

1-(4-Bromoanilino)-4-(4-pyridylmethyl)phthalazine;

1-(3-Chloro-4-methoxyanilino)-4-(4-pyridylmethyl)phthalazine;

1-(4-Cyanoanilino)-4-(4-pyridylmethyl)phthalazine;

1-(4-Methylanilino)-4-(4-pyridylmethyl)phthalazine;

1-(3-Chloro-4-fluoroanilino)-4-(4-pyridylmethyl)phthalazine;

1-(3-Methylanilino)-4-(4-pyridylmethyl)phthalazine;

and their pharmaceutically acceptable salts.

In compositions of the invention, the kinase inhibitor active ingredient is generally present in an amount from 1 to 95%, or 5 to 75%, or 10 to 50%, or 10 to 40%, or 10 to 30% by weight of the composition.

The compositions of the invention may also contain carriers or adjuncts such as those described in U.S. Pat. No. 6,258,812 or other conventional excipients. For oral administration, the composition may be contained in capsules, usually hard capsules of gelatin or soft capsules of gelatin mixed with a plasticizer such as glycerol or sorbitol, or may be used as a dispersion in an aqueous medium, such as water, saline solution or a mixture of water with another, water-miscible, pharmaceutically acceptable solvent, for example in an amount of 0.5 to 70, preferably 5 to 50% by weight, optionally together with a preservative, for example a conventional preservative such as a benzoate, particularly an ester of p-hydroxybenzoic acid such as the methyl, ethyl, n-propyl, n-butyl or benzyl ester thereof or the sodium salt of the ester and other excipients such as dispersing agents and suspending agents.

Pharmaceutical formulations suitable for parenteral administration may be formulated in aqueous solutions, preferably in physiologically compatible buffers such as Hanks' solution, Ringers solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms.

After pharmaceutical compositions have been prepared, they can be placed in an appropriate container and labeled for treatment of an indicated condition. For administration of the kinase inhibitors disclosed herein, such labeling would include amount, frequency, and method of administration.

Pharmaceutical compositions suitable for use in the invention include compositions where the active ingredients are contained in an effective amount to achieve the intended purpose. The determination of an effective dose is well within the capability of those skilled in the art.

Various biodegradable and biocompatible polymeric matrices comprising the kinase inhibitors set out above, including microcapsules, nanospheres, and implants, are useful in the practice of the present invention.

Microspheres are fine spherical particles containing active drugs. They are differentiated from nanospheres primarily by the size of the particle; microspheres have a diameter of less than approximately 1000 μm, while nanospheres are submicronic (<1 μm). Microsphere systems contain either homogeneous monolithic microspheres, in which the drug is dissolved or dispersed homogeneously throughout the polymer matrix, or reservoir-type microspheres, in which the drug is surrounded by the polymer matrix membrane shell. Monolithic and reservoir systems can also be combined. For instance, active drug can be dispersed within, or adsorbed onto, the polymer surface in a reservoir-type microsphere.

Biodegradable polymers can consist of either natural or synthetic materials that vary in purity. Natural polymers include polypeptides and proteins (e.g., albumin, fibrinogen, gelatin, collagen), polysaccharides (e.g., hyaluronic acid, starch, chitosan), and virus envelopes and living cells (e.g., erythrocytes, fibroblasts, myoblasts). Natural materials require cross-linking in the microencapsulation process, leading to the denaturation of the polymer and the embedded drug. As a result, synthetic polymers are most commonly used. Frequently used synthetic polymers include poly(-hydroxy) acids such as polylactic acid (PLA), polyhydroxybutryic acid, and copoly (lactic/glycolic) acid (PLGA). These compounds are biocompatible, lack immunogenicity, and have physical properties that permit them to be easily shaped (to control the bioerosion rate).

Useful polymers include thermogels, i.e., hydrogels that alter their viscosity in response to changes in temperature. Such thermogels are known in the art and can contain, inter alia, an entangled network of two randomly grafted polymers, e.g., a network of poly(acrylic acid) and a triblock copolymer containing poly(propylene oxide) (“PPO”) and polyethylene oxide) (“PEO”) segments in the sequence PEO-PPO-PEO. This family of polymers goes by the trade name Pluronic polyols. Another Pluronic-based thermogel comprises Pluronic side chains grafted onto a bioadhesive backbone of either poly(acrylic acid) or chitosan. The thermogels useful in the invention are those that are liquid at room temperature, but that form gels at the normal temperature of the human body, i.e., about 37° C.

Colloidal particulate carriers can also be used in the methods of the present invention for delivering kinase inhibitor drugs. Liposomes are the preferred colloidal vehicle, and are composed of a phospholipid bilayer that may act as a carrier for both hydrophilic and hydrophobic medications. Liposomes can be made from, e.g., neutral lipids, charged phospholipids, and cholesterol. The addition of an amphophilic polymer such as polyethylene glycol (PEG) onto the surface of a liposome can slow the clearance of liposomes.

The disclosure of all patents, publications, (including published patent applications), and database accession numbers and depository accession numbers referenced in this specification are specifically incorporated herein by reference in their entirety to the same extent as if each such individual patent, publication, and database accession number, and depository accession number are specifically and individually indicated to be incorporated by reference.

The present invention is further illustrated with the following examples. However, the example is not to be construed as limiting the invention thereto.

### EXAMPLE 1

Intraperitoneally administered radiolabeled tracer can be used to quantitate BRB breakdown in mice exposed to vascular endothelial growth factor (VEGF). This technique is particularly advantageous with neonatal mice because of the technical difficulty involved with intravascular injections. Also, the comparison of retinas from injected and control eyes to tissues without a blood-tissue barrier corrects for variations in the amount of isotope injected or absorbed into the circulation.

Ten six- to seven-week-old C57B/J6 mice (Jackson Labs, Bar Harbor, Me.) are weighed to determine the proper dosage of anesthesia and tracer required. Mice are anesthetized with a solution containing 25 mg/kg ketamine (Ketaset; Fort Dodge Animal Health, Fort Dodge, Iowa) and 4 mg/kg rompun (Tranquived; Vedco, St. Joseph, Mo.) diluted 1:10 in saline prior to injection. Five mice are subjected to gavage with 50 mg/kg 4-pyridylmethyl-phthalazine derivative for three days prior to intravitreal administration of VEGF; the other five mice are subjected to gavage with vehicle.

Tropicamide (1%) is used to dilate the pupils of anesthetized mice. Under a dissecting microscope, a Harvard injector (PLI 100) is used with a glass pipet pulled to a diameter of 13-20 μm to inject 1 μl of saline containing VEGF (R&D Systems, Minneapolis, Minn.). The eyes are entered posterior to the limbus, directing the pipet toward the optic nerve. The solution is then deposited just in front of the retina without inflicting any damage to the lens. The eye are injected with VEGF at a concentration of 10⁻⁶ M. BRB integrity is assayed at 6 hours by administration of [³H]-mannitol tracer at 1 μCi/g. Sixty minutes after injection, the mice are injected with metofane and euthanized by cervical dislocation.

Retinas are rapidly removed by grasping behind the globe with Crile forceps and cutting across the anterior chamber with a Weck razor blade. Retinas are isolated by quickly removing the contents of the posterior chamber with a curved Crile forcep, dissecting it free from RPE, lens, and vitreous, and placing it in a pre-weighed scintillation vial within 30 seconds of sacrifice. The thoracic cavity is opened at the xyphoid process, cutting through the sternum, and the left superior lobe of the lung is removed and placed in a pre-weighed glass vial, being careful not to nick the heart or pulmonary vein. The left kidney is removed dorsally and excess fat is trimmed away prior to placing it in another pre-weighed scintillation vial. The tissue is allowed to dry within the vials for 20 minutes, following which the vials containing tissue are weighed to determine the net weight of the tissue.

One ml NCSII (Amersham, Chicago, Ill.) solubilizing solution is added to each vial and the vials are incubated overnight in a 50° C. water bath. The solubilized tissue is brought to room temperature and decolorized with 20% benzoyl peroxide in toluene in a 50° C. water bath. The tissues are again brought to room temperature and 5 ml Cytoscint ES (ICN, Aurora, Ohio) and 30 μl glacial acetic acid is added. The vials are stored for several hours in darkness at 4° C. to decrease chemiluminescence. The specimens are counted on a Wallace 1409 Liquid Scintillation Counter (Gaithersburg, :Md.).

The counts per mg tissue are determined for treated (with 4-pyridylmethyl-phthalazine derivative) and untreated retina, lung, and kidney. Ratios of these values for treated or untreated retina/lung or kidney and for lung/kidney are calculated. The lung/kidney ratios obtained for retinas from animals treated with 4-pyridylmethyl-phthalazine are compared to those gavaged with vehicle only using Student's t test with pooled variances.

It is found that the values of the retina/lung ratio (cpm per mg of retina/cpm per mg of lung) and the retina/kidney ratio are significantly reduced in the VEGF/4-pyridylmethyl-phthalazine-treated mice as compared to the VEGF/vehicle-treated mice. The retina/lung ratio in the VEGF/vehicle-treated mice is 0.8785±0.0946 versus 0.6326±0.0656 in the VEGF/4-pyridylmethyl-phthalazine-treated mice (Student-t value 0.0467). The retina/kidney ratio in the VEGF/vehicle-treated mice is 0.6806±0.1060 versus 0.3881±0.0436 in the VEGF/4-pyridylmethyl-phthalazine-treated mice (Student-t value 0.0201).

For comparison, control studies are done without administration of VEGF (i.e., no VEGF but administration by gavage of the vehicle for 4-pyridylmethyl-phthalazine). The retina/lung ratio in the vehicle-only-treated mice is 0.422125±0.0771. The retina/kidney ratio in the vehicle-only-treated mice is 0.332125±0.0784. Both values are significantly different from those obtained from the VEGF and vehicle-treated mice.

Thus, there is significantly less radioactivity in the retinas of VEGF-treated mice treated with 4-pyridylmethyl-phthalazine than in the retinas of untreated mice, indicating that there is significantly less leakage of radioactive tracer out of the retinal blood vessels of the 4-pyridylmethyl-phthalazine-treated mice, demonstrating the utility of 4-pyridylmethyl-phthalazine in decreasing leakage from retinal capillaries of the BRB in response to a known stimulator of such leakage, VEGF.

1. A method for decreasing or attenuating an increase in capillary permeability in the retina in a subject in need of such treatment, comprising administering a composition comprising an amount of a phthalazine derivative or a salt thereof to a subject suffering from excessive or pathological capillary permeability in the retina, the amount of phthalazine derivative or salt thereof being effective to decrease the permeability of capillaries in the retina of the subject.

2. The method of claim 1 wherein the subject is suffering from clinically significant macular edema.

3. The method of claim 1 wherein the subject is suffering from macular edema which is not clinically significant.

4. The method of claim 2 wherein the subject is suffering from visual acuity loss.

5. The method of claim 1, wherein the effective amount is between about 500 and about 2000 milligrams per day.

6. The method of claim 5, wherein the effective amount is between about 1000 and about 2000 milligrams per day, and the phthalazine derivative or a salt thereof is 1-(4-Chloroanilino)-4-(4-pyridylmethyl)phthalazine.

7. The method of claim 6, wherein the total effective amount is delivered to the subject in a single administration at a single time point during the day.

8. The method of claim 6, wherein the total effective amount is delivered to the subject in multiple administrations at multiple time points during the day.

9. The method of claim 1, wherein the effective amount is administered topically to the eye.

10. The method of claim 1, wherein the effective amount is administered intravitreally to the eye.

11. The method of claim 1, wherein the effective amount is administered subconjunctivally to the eye.

12. The method of claim 1, wherein the effective amount is administered periorbitally to the eye.

13. A method for decreasing capillary permeability in the retina in a subject in need of such treatment according to claim 1, wherein the subject is suffering from macular edema but does not also suffer from diabetes.

14. The method of claim 14 wherein said macular edema is caused by a member selected from the group consisting of branch retinal vein occlusion, pseudophakic cystoid macular edema, aphakic cystoid macular edema, intermediate uveitis, idiopathic retinal telangiectasia, vitreomacular traction syndrome, and dominantly inherited cystoid macular edema.

15. A method for treating a subject suffering from diabetic neuropathy comprising administering an effective amount of a composition comprising an amount of a phthalazine derivative or a salt thereof to a subject suffering from diabetic neuropathy to ameliorate at least one symptom of said diabetic neuropathy.

17-23. (canceled)
